(19) United States
(12) Patent Application Publication
(10) Pub. No.: US 2005/0273088 A1
(43) Pub. Date: Dec. 8, 2005

(54) Myopia correction enhancing biodynamic ablation

(76) Inventors: Gerhard Youssefi, Landshut (DE); Friedrich Moritz, Munchen (DE)

Correspondence Address: Craig E. Larson;Bausch & Lomb Incorporated, One Bausch & Lomb Place, Rochester, NY, 14604, US

(21) Appl. No.: 10/519,368

(22) PCT Filed: Jun. 26, 2003
(86) PCT No.: PCT/EP03/06778

§ 371 Date: Jul. 14, 2005

Related U.S. Application Data
(60) Provisional application No. 60/392,510, filed on Jun. 27, 2002.

(51) Int. Cl. A61B 18/20

(52) U.S. Cl. 606/5

(57) ABSTRACT

This invention is directed to a method for providing a LASIK or a LASEK myopia vision correction, and to a medium that has stored therein an instruction for directing a laser vision correcting laser platform to deliver a controlled biodynamic ablation according to the invention. A known biodynamic response of the eye is induced by performing a controlled laser ablation in a cornea of the eye outside of an optical zone for the nominal ablation in a laser vision correction surgery. An ablation ring, or portion thereof, outside of the optical zone produces a biodynamic flattening of the central region of the cornea which in turn provides for a decreased depth of volumetric ablation to correct a myopic refractive defect of the eye. Such controlled biodynamic flattening may provide the opportunity for laser vision correction in patients whose corneas would otherwise be too thin post-operatively to warrant laser vision correction.

### BACKGROUND OF THE INVENTION

1. Field of the Invention

The invention is generally directed to the field of laser vision correction and, more particularly, to a method and device for inducing and utilizing a corneal biodynamic effect for improving laser vision correction.

2. Description of Related Art

The field of laser vision correction currently offers several types of procedures for correcting or improving refractive defects by laser photoablation of the corneal surface. These procedures include PRK, LASIK, and LASEK, which are typically used to correct myopic and hyperopic defects with or without astigmatism, and in some cases provide customized treatments to address the higher order aberrations of the eye.

The evolution of laser technology and its use in the vision correction field has contributed significantly to the state of the art of laser vision correction. Ten years ago, broad-beam lasers utilizing variable diaphragms and/or masks were used to treat myopia by flattening the central region of the cornea and, to a lesser extent, for the treatment of hyperopia. Shortly thereafter, scanning beam technology contributed to the development of small-beam, scanning-type laser systems, and flying-spot beam delivery systems producing laser beams from 0.5 mm to 2.0 mm on the corneal surface. These smaller beam sizes in combination with optimized scanning patterns, and now with much higher laser pulse repetition rates, define the landscape for photoablative contouring of the cornea.

A well known technique for delivering a conventional myopic LASIK treatment is the Planoscan® ablation algorithm delivered by the Technolas 217A® laser system (Bausch & Lomb Incorporated, Rochester, N.Y.). In this system, selected scanning patterns of a 2 mm diameter laser beam are used to ablate the corneal surface. The interested reader is referred to U.S. Pat. Nos. 6,090,100 and 5,683,379 which are herein incorporated by reference in its entirety to the full extent allowed by applicable laws and rules. Recently, Bausch & Lomb Incorporated introduced the Zypotix® vision correction system incorporating the Zywave® Hartmann-Shack wavefront sensor and the 217Z® excimer laser system which delivers 1 mm to 2 mm diameter, truncated Gaussian beams onto the cornea for customized laser vision correction.

A long-standing concern held by laser manufacturers and surgeons alike, is the amount of corneal tissue ablated by any laser vision correction. In general terms, a surgeon intending to perform a myopia correction to a patient's eye will determine the amount of refractive correction necessary to correct the person's vision (typically measured in diopters), and also determine the optical zone (OZ) over which the ablation should occur. The OZ typically ranges from about 3 mm to 7 mm depending upon a variety of factors well appreciated by those skilled in the art. Once the desired refractive correction and the optical zone size are determined, the maximum central ablation depth required for the correction will be known. Corneal ablation will be contraindicated when the corneal thickness remaining after the removal of corneal tissue by the ablation procedure will be less than what is considered to be a minimum residual thickness under a reasonable standard of care. Typically, no less than 200 microns and, preferably, about 250 microns is the minimum tolerable residual corneal thickness. One solution is to decrease the OZ size; however, one cause of post-LASIK spherical aberration resulting in glare and halo effects in low-light conditions is believed to be due to an ablated OZ that is smaller than the patient's pupil in low light conditions.

It is also recognized that the response of the eye to trauma due, for example, to a LASIK keratectomy or the ablation of corneal tissue, adds a degree of uncertainty to the effect induced by the traumatic cause. Thus, changes in the structural integrity of the eye produce what will be referred to herein as biodynamic responses that manifest themselves in the form of corneal flattening, corneal thickening, regression, wound healing responses, and in other physical ways that are not yet fully understood.

In view of the foregoing, the inventors have recognized a need for overcoming the limitations and concerns discussed above in providing improved vision through laser vision correction.

### SUMMARY OF THE INVENTION

An embodiment of the invention is directed to a method for a LASIK or a LASEK myopia (with or without astigmatism) laser vision correction, including the control and improvement thereof. The method generally relies on a corneal biodynamic effect to reduce the amount of tissue ablation, i.e., ablation depth, as a function of increased optical zone size. According to the invention, a corneal biodynamic effect is induced which results in a flattening of the central corneal region, By flattening the cornea in a controlled manner, a shallower myopia correcting ablation can be performed over an optical zone area than would occur over the same optical zone area if the cornea were not flattened from its original shape. In a preferred aspect, the trauma inflicted to the eye is a biodynamic ablation in the form of at least one or more portions of, or a complete, ring or annulus. The biodynamic ring may be circular or non-circular (i.e., elliptical or other shape). In this aspect, the ring or annulus of ablated corneal tissue is outside of and surrounding the optical zone. The parameters of the biodynamic ring, particularly the distance from the optical zone edge, and the width and depth of the ring, all of which are variable as a function of biodynamic ablation location, will produce a controlled biodynamic effect that will be advantageous for reducing the ablation depth of corneal tissue in the optical zone to effect a myopia correction.

Another embodiment of the invention is directed to an improved device readable medium having stored therein an executable instruction or instruction code for directing an ophthalmic vision correcting laser platform to deliver a myopia correcting nominal ablation in an optical zone of a corneal surface, where the improvement comprises an executable instruction or instruction code stored in the medium for directing the ophthalmic vision correcting laser platform to deliver a myopia correction enhancing biodynamic ablation in the corneal surface outside of the optical zone.

The objects and advantages of the invention will be further appreciated in view of the detailed description and drawings that follow, and by the appended claims which define the invention.

### BRIEF DESCRIPTION OF THE DRAWINGS

The accompanying drawings, which are incorporated in and constitute a part of this specification illustrate embodiments of the present invention and, together with the description, serve to explain the objects, advantages and principals of the invention. In the drawings,

FIG. 1 is a schematic front view of an eye showing a biodynamic ablation region according to an embodiment of the invention;

FIG. 2 is an enlargement of a central portion of FIG. 1 showing a more detailed representation of the biodynamic ablation region;

FIG. 3 is a schematic cross-sectional view of the biodynamic ablation according to a preferred embodiment of the invention;

FIG. 4 is a schematic cross-sectional view of a corneal profile showing the effect on the profile due to the biodynamic ablation according to an embodiment of the invention;

FIG. 5 is an illustration of a laser beam profile associated with a preferred embodiment of the invention;

FIG. 6 is an enlarged photocopy of a laser beam profile shaping aperture associated with a preferred embodiment of the invention; and

FIG. 7 is a schematic illustration of a device embodiment of the invention.

### DETAILED DESCRIPTION OF A PREFERRED EMBODIMENT

The invention is directed to a method for a LASIK or a LASEK myopia (with or without astigmatism) laser vision correction, and to a computer or device readable medium having stored therein an executable instruction or instruction code for directing an ophthalmic vision correcting laser platform to perform a myopia correction enhancing biodynamic ablation according to an embodiment of the invention.

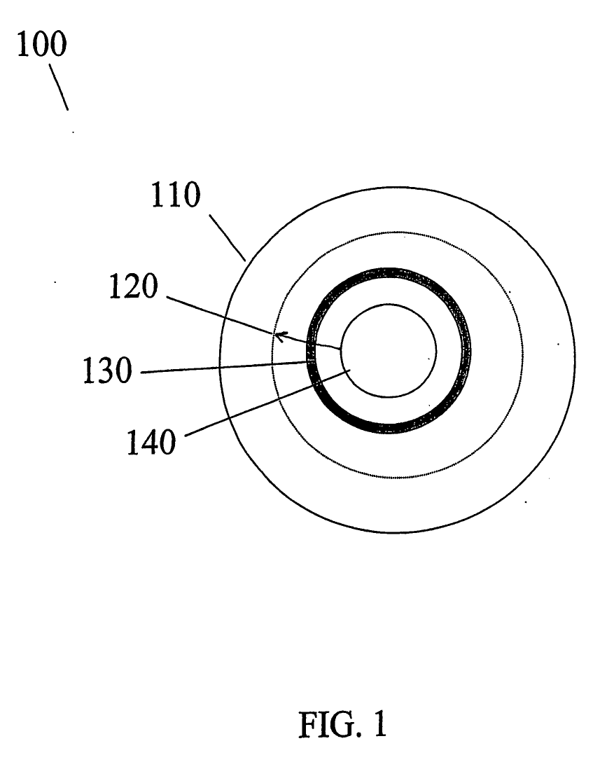

FIG. 1 schematically shows a front view of an eye 100 including an optical zone (OZ) 140 of the eye, and the outer boundary 110 of the iris of the eye. When LASIK or LASEK corrective surgery is performed on an eye, the actual vision correcting ablation which is referred to herein as the nominal ablation, is typically performed over a region of the pupil (dilated or undilated) referred to as the optical zone 140. A transition zone 120 typically lies outside of and immediately adjacent to the optical zone and defines a boundary between the ablated and non-ablated areas of the cornea. According to an embodiment of the invention, a controlled biodynamic response will be induced in the eye by inflicting a controlled trauma represented as 130 in the exposed corneal surface outside of the optical zone 140 of the eye and preferably within the transition zone 120.

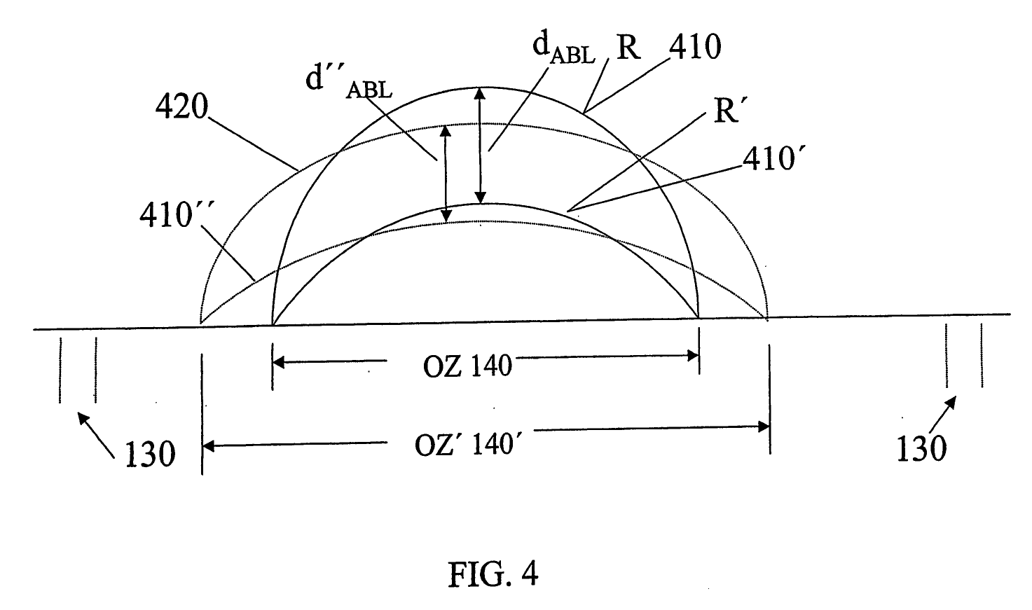

The most commonly occurring refractive defect in the general population is called myopia. Myopia, or nearsightedness, is due to a corneal shape that is too prolate or bullet shaped in profile, such that images are formed in front of the retina instead of at the retinal plane. FIG. 4 schematically shows a pre-operative corneal profile 410 for a typically myopic eye. It will be appreciated that the drawings referred to herein are not to scale but are intended to illustrate embodiments of the invention as defined herein and in the appended claims.

It is well appreciated by those skilled in the art that to correct for myopia, the pre-operative corneal profile 410 having a pre-operative radius R must be flattened over the optical zone 140. A desired post-operative surface 410′ having a larger radius R′ necessary to correct the myopic defect determines the ablation depth, d_{abl}, for the nominal volumetric ablation of corneal tissue, as shown in FIG. 4. It often occurs, however, that the necessary ablation depth d_{abl} results in a residual, post-operative corneal thickness that is not thick enough (typically 200-250 microns) to maintain the structural integrity of the cornea, and/or meet a reasonable standard of care in the medical community. If the depth of ablation d_{abl} decreases and R′ is maintained, the optical zone would shrink correspondingly. This becomes problematic since one cause of poor low-light vision, manifested by glare and halo effects, is believed to be due to a nominal ablation in an optical zone that is smaller than the (dilated) pupil size in the low-light environment. As such, laser corrective surgery may not be an option under these circumstances.

Advantageously, it has been found that by inflicting a controlled trauma to a selected region of the cornea, a controlled biodynamic response of the eye can be induced that is manifested by a flattening of the corneal profile at least over a central region of the cornea. The biodynamic flattening, represented by dotted line 420 in FIG. 4 is preferably induced by ablating a ring of tissue illustrated at 130 in FIGS. 1, 2, and 4. As further shown in FIG. 4, the biodynamic flattening of the cornea illustrated at 420 increases the optical zone size to the dimensions schematically shown at 140′ (OZ′). Now the calculated post-operative radius of curvature R′ can be created by surface profile 410″ over the new optical zone 140′ by ablating a corneal tissue volume depth d″_{abl} that is less than the original nominal ablation depth d_{abl}.

Although the biodynamic ablation according to a preferred aspect of the invention as set forth below is described in the form of a circular annulus or ring, it is to be understood that the ring may be elliptical or otherwise shaped, and may constitute only a portion, or discontinuous portions, of any such ring ablation. Biodynamic ring shape and location, including ring width and depth, may depend upon corneal thickness and/or refractive properties (e.g., astigmatism), or other factors. Thus, the illustrative description set forth below is not intended to limit the scope of the invention in any manner, but only to simplify the understanding of the invention described and claimed herein.

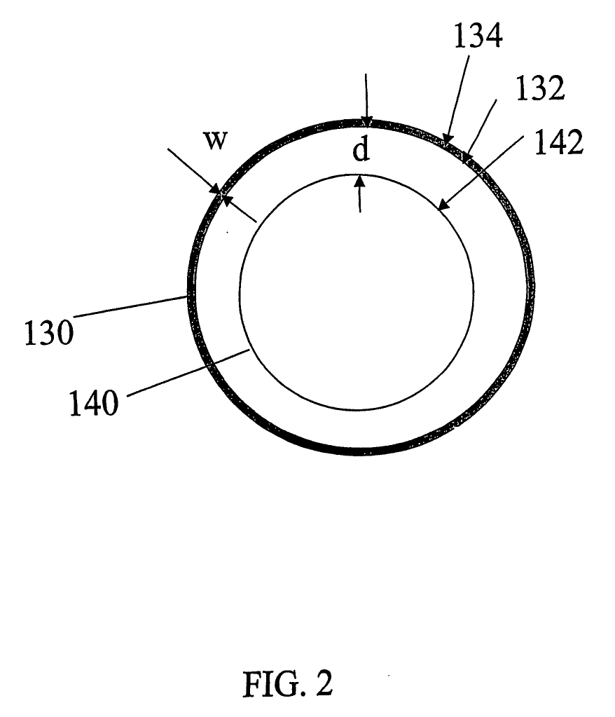

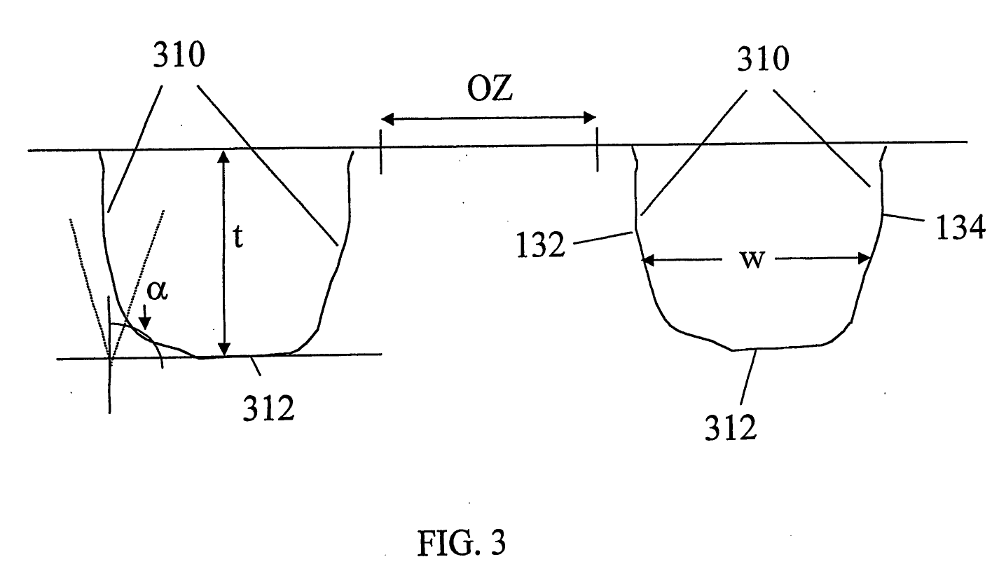

As illustrated in FIGS. 1 and 2, the biodynamic ring 130 has an inner boundary edge 132 and an outer boundary edge 134 defining a ring width, w. The inner boundary of the biodynamic ring 132 is adjacent an outer boundary of the nominal ablation optical zone 140 and separated therefrom by a minimum distance, d, as shown in FIG. 2. The distance d is preferably between about 200 microns to 600 microns. As illustrated in the schematic cross-section in FIG. 3, the biodynamic ring 130 has an ablation depth, t. In a preferred aspect, the width, w, of the biodynamic ring is nominally 1 mm, and the depth of ablation, t, is between about 10 microns to 70 microns.

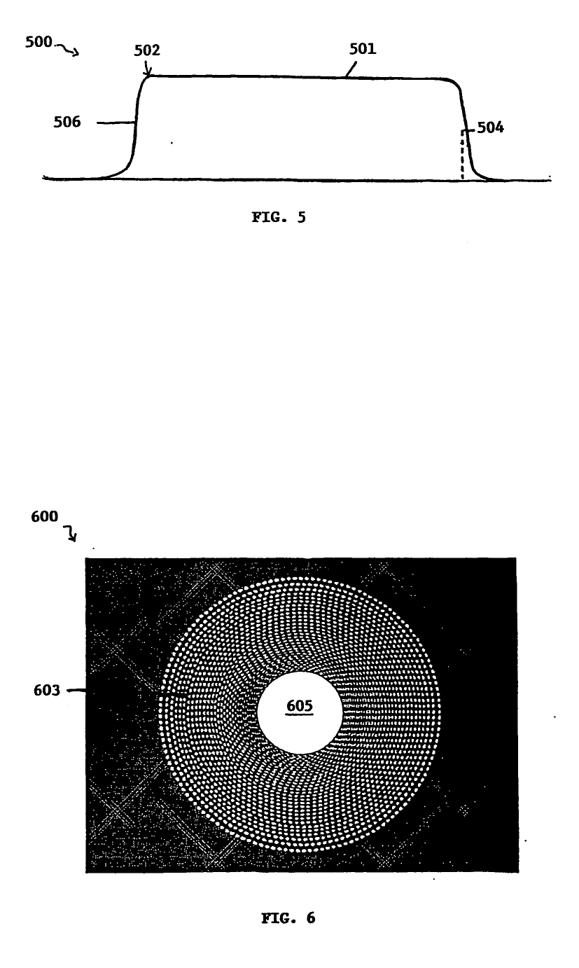

It is preferred that the ablation channel formed by the biodynamic ring have sidewalls 310 that are nominally perpendicular to the floor surface 312 of the channel. This is illustrated by the angle, α, shown in FIG. 3. Such a controlled ablation ring profile can be produced by a laser beam at the target surface having an energy profile 500 shown schematically in FIG. 5. FIG. 5 shows what is referred to herein as a “soft-spot” profile, which is described in detail in co-owned published application WO 01/28478, the description of which is incorporated by reference herein in its entirety to the extent allowed by applicable laws and rules. As illustrated, the soft-spot profile 500 is defined as having a center portion 501 that is flat or substantially flat, and an edge 502 of the profile is continuous with the center portion and is rounded. The center portion 501 is preferably symmetric about the radius of the profile and extends across about 60 to 80 percent and more preferably, across about 65 to 70 percent of the total profile 500. At a certain point, such as an intensity threshold point 504 at which the eye tissue ablation intensity threshold is no longer reached, the profile 500 preferably quickly drops off or diminishes as a substantially square, vertical, or truncated edge 506. The ablation threshold or any variations in it are known in the art. The amount of energy falling below the threshold for ablation is preferably about 5 percent or less of the total energy encompassed by the profile 500. The profile 500 is non-Gaussian, between square and Gaussian shaped, referred to herein as a truncated Gaussian. Referring to FIG. 6, the soft-spot energy profile 500 can be produced by what is referred to herein as a soft-spot aperture 600. The aperture 600 comprises a larger, central, directly transmitting aperture portion 605 surrounded by a plurality of smaller subapertures 603 that diffractively transmit the laser beam. These apertures can be obtained from Fraunhofer Institut Siliziumtechnologie, Faunhoferstrabe 1, D-25524 Itzehoe, Germany, and from others, and are further described in detail in the published application referred to immediately above.

Other beam energy profiles will produce corresponding different ablation channel profiles. The determination of the specific parameters associated with the size, shape, and placement of the biodynamic ring, will benefit from continued modeling refinements, and further empirical analysis of statistically significant population groups will lead to more accurate relationships between biodynamic ablation parameters and desired biodynamic responses.

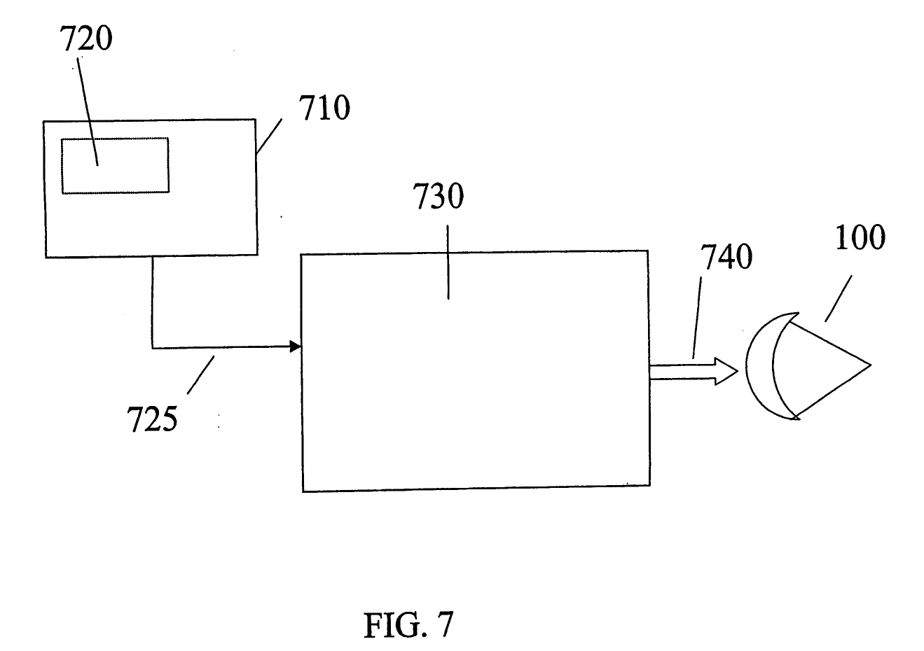

Another embodiment according to the invention, shown with reference to FIG. 7, is directed to an improved device readable medium 710 having stored therein an executable instruction for directing an ophthalmic laser platform 730 to deliver a myopia correcting nominal ablation in an optical zone of the corneal surface, where the improvement is directed an executable instruction 720 stored in the medium 710 for directing the laser platform to deliver a myopia correction enhancing biodynamic ablation 130 in the corneal surface outside of the optical zone 140 as described hereinabove. The particular architecture of the executable instruction can take various forms. In a preferred exemplary aspect, an enablement type card used with the laser platform may have a data storage capability comprising software that is downloadable by the laser platform instructing it to deliver the biodynamic ablation. In an alternative aspect, the medium may contain a code that can match a pre-programmed instructional routine resident in, or external to, the laser platform, whereupon matching the instruction code with the resident instruction will enable the laser platform to execute the biodynamic ablation. These foregoing aspects are in no way intended to limit the invention as described but merely to set forth exemplary implementations of the invention. A further description of a device readable medium and associated instructional code relating to the control of laser vision correction is presented in co-pending application Ser. No. 10/184,441 entitled Laser Vision Correction Apparatus and Control Method, filed concurrently and commonly owned with the instant application

Notwithstanding the preferred embodiments specifically illustrated and described herein, it will be appreciated that various modifications and variations of the instant invention are possible in light of the description set forth above and the appended claims, without departing from the spirit and scope of the invention.

1. An apparatus for laser vision correction comprising means for controlling the apparatus to deliver a myopia correcting nominal laser ablation in an optical zone identified for the myopia correcting nominal ablation of an exposed corneal surface of an eye, characterized in that:
the apparatus is further controlled to deliver a laser ablation in a region outside of the identified optical zone and separated therefrom by a minimum distance so as to create a central flattening of the corneal surface via a controlled biodynamic response to the exposed corneal surface outside of the identified optical zone.

2. The apparatus of claim 1, wherein the laser ablation in a region outside of the identified optical zone is at least part of an ablation ring.

3. The apparatus of claim 2, wherein the at least part of an ablation ring is either circular or acircular in shape.

4. The apparatus of claim 2, wherein the at least part of an ablation ring has an inner boundary adjacent an outer boundary of the identified optical zone.

5. The apparatus of claim 4, wherein the inner boundary of the at least part of the ablation ring begins at a distance, d, from the outer boundary of the identified optical zone, where 200 μm≦d≦600 μm.

6. The apparatus of claim 2, wherein the at least part of the ablation ring has a depth, t, where 10 μm≦t≦70 μm, and a width, w.

7. The apparatus of claim 6, wherein t and w are variable as a function of the biodynamic ablation location on the cornea.

8. The apparatus of claim 6, wherein w is a function of a laser beam diameter on the cornea.

9. The apparatus of claim 6, wherein w has a nominal value of about 1 mm.

10. The apparatus of claim 1, wherein the laser ablation in a region outside of the identified optical zone lies within at least part of a transition zone of the nominal ablation.

11. The apparatus of claim 1, wherein the means for providing the controlled biodynamic response creates a tissue ablation volume for a desired refractive correction that is less than a corresponding tissue ablation volume for the desired refractive correction in the absence of the controlled biodynamic response.

12. The apparatus of claim 11, wherein the lessened tissue ablation volume has a smaller ablation depth over the optical zone than a corresponding ablation depth over the optical zone in the absence of the controlled biodynamic response.

13. The apparatus of claim 1, wherein the means for providing the controlled biodynamic response empirically determines the controlled biodynamic response from a statistically significant population.

14. The apparatus of claim 1, wherein the means for providing the controlled biodynamic response delivers a plurality of photoablative light pulses onto the corneal surface, all of which have only a 1 mm diameter.

15. The apparatus of claim 14, wherein the plurality of photoablative light pulses have a direct aperture transmission portion and a diffractive aperture transmission portion so as to produce a soft-spot beam intensity profile.

16. The apparatus of any of claims 1 to 15, further characterized by:
a medium readable by the apparatus including an executable instruction for directing the apparatus to deliver the myopia correcting nominal ablation in the identified optical zone of the corneal surface, and further including an executable instruction for directing the apparatus to deliver a myopia correction enhancing biodynamic ablation in the corneal surface outside of the identified optical zone.
